(19) United States
(12) Patent Application Publication
(10) Pub. No.: US 2013/0184476 A1
(43) Pub. Date: Jul. 18, 2013

(54) PREPARATION OF LUBIPROSTONE

(71) Applicants: Mark Jackson, Milton (GB); Vilas Hareshwar Dahanukar, Hyderabad (IN); Suju Chuttippari Joseph, Trivandrum (IN); Vishnu Vardhana Verma Reddy Eda, Hyderabad (IN); Sandip Khobare Ramdas, Ahmednagar (IN)

(72) Inventors: Mark Jackson, Milton (GB); Vilas Hareshwar Dahanukar, Hyderabad (IN); Suju Chuttippari Joseph, Trivandrum (IN); Vishnu Vardhana Verma Reddy Eda, Hyderabad (IN); Sandip Khobare Ramdas, Ahmednagar (IN)

(21) Appl. No.: 13/546,067

(22) Filed: Jul. 11, 2012

Related U.S. Application Data
(60) Provisional application No. 61/527,737, filed on Aug. 26, 2011.

(30) Foreign Application Priority Data
Jul. 13, 2011 (IN) 2389/CHE/2011

(51) Int. Cl. C07D 311/94

(52) U.S. Cl. CPC C07D 311/94
USPC 549/396

(57) ABSTRACT

Aspects of the present application relate to process for the preparation of lubiprostone.

This application claims priority to Indian Provisional Application 2389/CHE/2011, filed on Jul. 13, 2011 and U.S. Provisional Application No. 61/527,737, filed on filed on Aug. 26, 2011; all of which are hereby incorporated by reference in their entirety.

### INTRODUCTION

Aspects of the present application relates to process for the preparation of lubiprostone and intermediates thereof.

Lubiprostone is chemically described as (−)-7-[(2R,4aR,5R,7aR)-2-(1,1-difluoropentyl)-2-hydroxy-6-oxooctahydrocyclopenta[b]pyran-5-yl]heptanoic acid. It has the structure of formula (I).

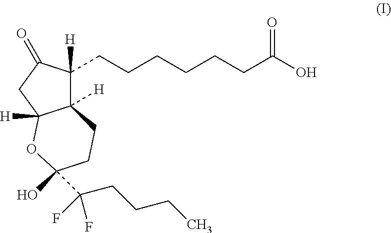 [SMILES: CCCCC(F)(F)[C@@]1(O)CC[C@H]2[C@@H](CC(=O)[C@@H]2CCCCCCC(=O)O)O1]

Lubiprostone is a locally acting chloride channel activator and is indicated for: (a) treatment of chronic idiopathic constipation in adults; and (b) treatment of irritable bowel syndrome with constipation (IBS-C) in women 18 years old; and is contained in products sold as Amitiza®.

U.S. Pat. No. 5,284,858 discloses 13,14-dihydro-15-keto-16,16-difluoro-prostaglandins. U.S. Pat. No. 7,355,064 discloses a process for the preparation of 15-keto-prostaglandin E derivatives by hydrolyzing or deprotecting the intermediate of a 15-keto-prostaglandin E derivative having a protected hydroxyl group in the presence of a phosphoric acid compound.

There remains a need to provide improved process for the preparation of lubiprostone and its intermediates that are cost-effective and environment friendly.

### SUMMARY

In an aspect, the present application provides an improved process for the preparation of lubiprostone of formula (I):

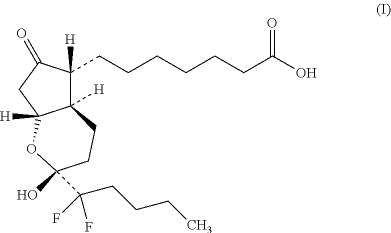

which includes one or more of the following steps, individually or in the sequence recited:

(a) reacting a compound of formula (II) with a reagent to provide a compound of formula (III);

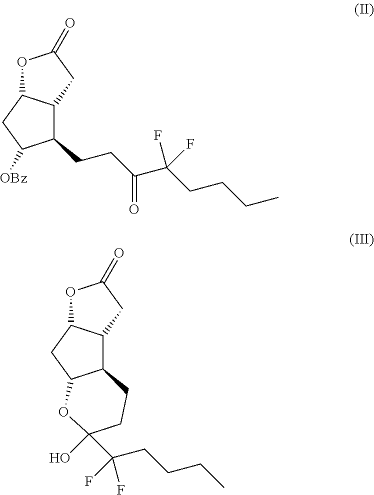

(b) reacting the compound of formula (III) with dihydropyran to provide a compound of formula (IV);

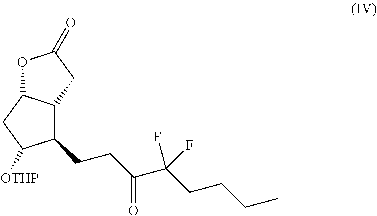

(c) converting a compound of formula (IV) in to a compound of formula (V);

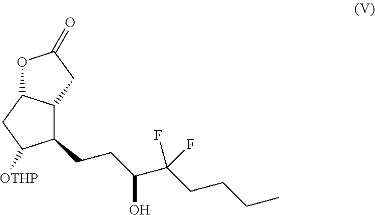

(d) converting a compound of formula (V) in to a compound of formula (VI);

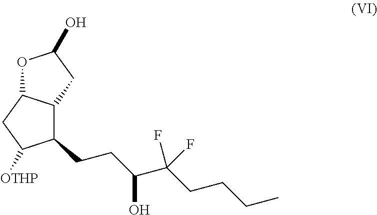

(e) reacting a compound of formula (VI) with (4-carboxybutyl)triphenylphosphonium bromide to provide a compound of formula (VII);

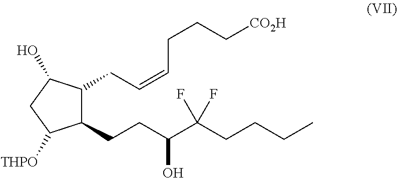

(f) converting a compound of formula (VII) in to a compound of formula (VIII);

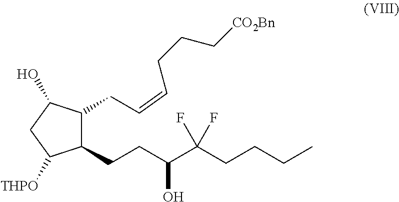

(g) reacting a compound of formula (VIII) with oxidizing agent to provide a compound of formula (IX);

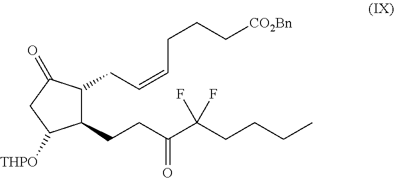

(h) reacting a compound of formula (IX) with an acid to provide a compound of formula (X); and

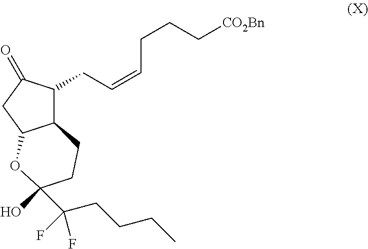

(i) converting a compound of formula (X) in to lubiprostone of formula (I).

In an aspect, the present application provides a process for the preparation of formula (XII):

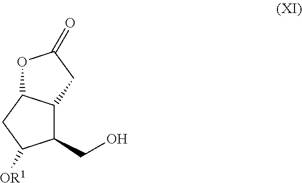

comprising reacting a compound of formula (XI) with a reagent to provide a compound of formula (XII):

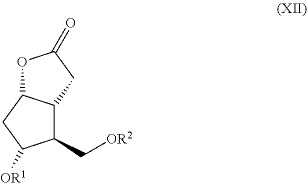

wherein each of R¹ and R² independently is a hydrogen or a protecting group for a hydroxyl group.

### DETAILED DESCRIPTION

In an aspect, the present application provides an improved process for the preparation of lubiprostone of formula (I):

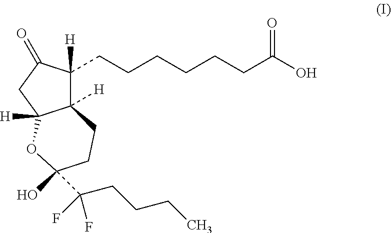

which includes one or more of the following steps, individually or in the sequence recited:

In an aspect, the present application provides an improved process for the preparation of lubiprostone of formula (I):

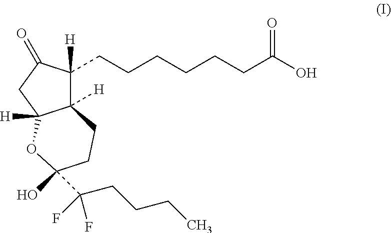

which includes one or more of the following steps, individually or in the sequence recited:

(a) reacting a compound of formula (II) with a reagent to provide a compound of formula (III);

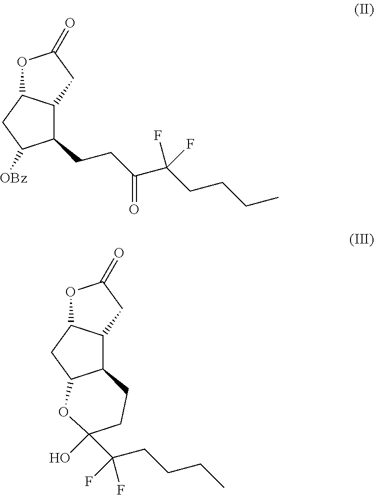

(b) reacting the compound of formula (III) with dihydropyran to provide a compound of formula (IV);

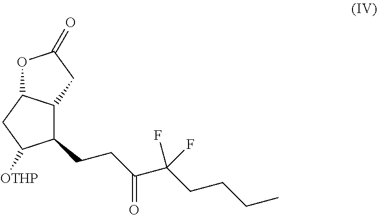

(c) converting a compound of formula (IV) in to a compound of formula (V);

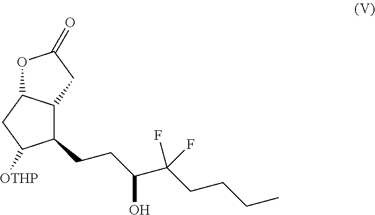

(d) converting a compound of formula (V) in to a compound of formula (VI);

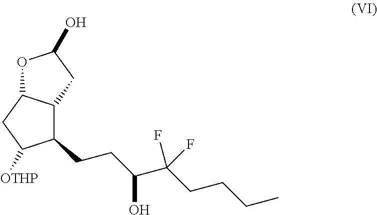

(e) reacting a compound of formula (VI) with (4-carboxybutyl)triphenylphosphonium bromide to provide a compound of formula (VII);

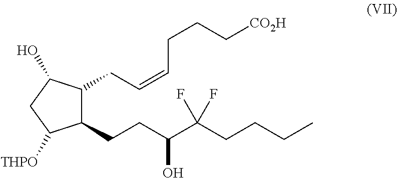

(f) converting a compound of formula (VII) in to a compound of formula (VIII);

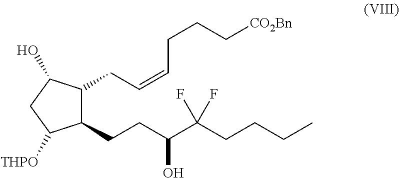

(g) reacting a compound of formula (VIII) with oxidizing agent to provide a compound of formula (IX);

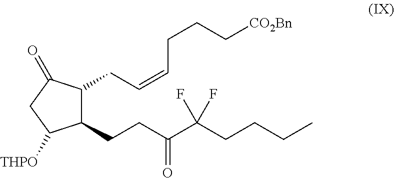

(h) reacting a compound of formula (IX) with an acid to provide a compound of formula (X); and

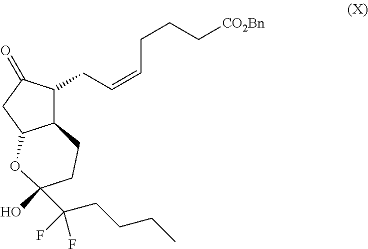

(i) converting a compound of formula (X) in to lubiprostone of formula (I).

Step (a) involves reacting a compound of formula (II) with a reagent to provide a compound of formula (III). Suitable reagents that may be used in step a) include, but are not limited to: alkali metal carbonates, such as, for example, sodium carbonate, potassium carbonate, lithium carbonate, cesium carbonate, or the like; alkaline earth metal carbonates, such as, for example, magnesium carbonate, calcium carbonate, or the like; alkali metal bicarbonates, such as, for example, sodium bicarbonate, potassium bicarbonate, or the like; alkali metal hydrides, such as, for example, sodium hydride, potassium hydride, or the like; sodamide; n-butyl lithium; lithium diisopropylamide; alkali metal hydroxides, such as, for example, lithium hydroxide, sodium hydroxide, potassium hydroxide, or cesium hydroxide; alkaline metal hydroxides, such as, for example, barium hydroxide, magnesium hydroxide, calcium hydroxide, or the like; or mixtures thereof; or any other suitable reagents.

Step (a) may be carried out in one or more suitable solvents. Suitable solvents that may be used in step a) include, but are not limited to, water; alcohol solvents; ketone solvents; halogenated solvents; ether solvents; aromatic hydrocarbon solvents; ester solvents; nitrile solvents; or mixtures thereof.

Suitable temperatures for the reaction of step (a) may be less than about 150° C., less than about 100° C., less than about 80° C., less than about 60° C., less than about 40° C., less than about 30° C., less than about 20° C., less than about 10° C., or any other suitable temperatures.

Optionally, the reaction mixture containing the compound of formula (III) obtained in step a), before or after conventional work-up, may be carried forward to step b) without isolating the compound.

Step (b) involves reacting the compound of formula (III) with dihydropyran to provide a compound of formula (IV).

Step (b) may be carried out in the presence of a reagent. Suitable reagents that may be used in step b) include, but are not limited to: pyridinium-p-toluenesulfonate, pyridinium dichromate, carbonyldiimidazole, dicyclohexylcarbodiimide, or any other suitable coupling reagents.

Step (b) may be carried out in one or more suitable solvents. Suitable solvents that may be used in step (b) include, but are not limited to: water; halogenated hydrocarbon solvents; ester solvents; ether solvents; aromatic hydrocarbon solvents; nitrile solvents; or mixtures thereof.

Suitable temperatures for the reaction of step (b) may be less than about 150° C., less than about 100° C., less than about 80° C., less than about 60° C., less than about 40° C., less than about 30° C., less than about 20° C., less than about 10° C., or any other suitable temperatures.

Optionally, the reaction mixture containing the compound of formula (IV) obtained in step b), before or after conventional work-up, may be carried forward to step c) without isolating the compound.

Step (c) involves converting a compound of formula (IV) in to a compound of formula (V).

Step (c) may be carried out in the presence of a reducing agent. Suitable reducing agents that may be used in step c) include, but are not limited to: lithium aluminum hydride, sodium borohydride, lithium borohydride, potassium borohydride, NaCNBH₃, diisobutyl aluminium hydride (DIBAL), borane-dimethyl sulfide (BMS), borane-tetrahydrofuran (BTHF), or any other suitable reducing agents.

Step (c) may be carried out in one or more suitable solvents. Suitable solvents that may be used in step (c) include, but are not limited to: water, alcohol solvents; ketone solvents; halogenated solvents; ether solvents; hydrocarbon solvents; ester solvents; nitrile solvents, or mixtures thereof.

Suitable temperature for the reaction of step (c) may be less than about 80° C., less than about 60° C., less than about 40° C., less than about 30° C., less than about 20° C., less than about 10° C., less than about 0° C., less than about −10° C., less than about −20° C., less than about −30° C., or any other suitable temperatures.

Optionally, the reaction mixture containing the compound of formula (V) obtained in step c), before or after conventional work-up, may be carried forward to step d) without isolating the compound.

Step (d) involves converting a compound of formula (V) in to a compound of formula (VI).

Step (d) may be carried out in the presence of a reducing agent. Suitable reducing agents that may be used in step d) include, but are not limited to: diisobutyl aluminium hydride (DIBAL), lithium aluminum hydride, sodium borohydride, lithium borohydride, potassium borohydride, NaCNBH₃, borane-dimethyl sulfide (BMS), borane-tetrahydrofuran (BTHF), a combination thereof, or any other suitable reducing agents.

Step (d) may be carried out in one or more suitable solvents. Suitable solvents that may be used in step d) include, but are not limited to: water; hydrocarbon solvents; ester solvents; alcohol solvents; ketone solvents; halogenated solvents; ether solvents; nitrile solvents, or mixtures thereof.

Suitable temperatures for the reaction of step (d) may be less than about 30° C., less than about 20° C., less than about 10° C., less than about 0° C., less than about −10° C., less than about −20° C., less than about −30° C., less than about −40° C., less than about −50° C., less than about −60° C., less than about −70° C., less than about −80° C., or any other suitable temperatures.

Optionally, the reaction mixture containing the compound of formula (VI) obtained in step d), before or after conventional work-up, may be carried forward to step e) without isolating the compound.

Step (e) involves reacting a compound of formula (VI) with (4-carboxybutyl)triphenylphosphonium bromide in presence of a base to provide a compound of formula (VII).

Step (e) may be carried out in the presence of a base. Suitable bases that may be used in step e) include, but are not limited to: alkali metal alkoxides, such as, for example, potassium tertiary butoxide, sodium methoxide, potassium isopropoxide, or the like; alkali metal hydrides, such as, for example, sodium hydride, potassium hydride, calcium hydride, or the like; alkali metal hydroxides, such as, for example, lithium hydroxide, sodium hydroxide, potassium hydroxide, or cesium hydroxide; alkaline metal hydroxides, such as, for example, barium hydroxide, magnesium hydroxide, calcium hydroxide, or the like; or any other suitable bases.

Step (e) may be carried out in a suitable solvent. Suitable solvents that may be used in step e) include, but are not limited to: water; ether solvents; aliphatic or alicyclic hydrocarbon solvents; halogenated hydrocarbon solvents; aromatic hydrocarbon solvents; polar aprotic solvents, or any mixtures thereof.

Suitable temperatures for the reaction of step (e) may be less than about 80° C., less than about 60° C., less than about 40° C., less than about 30° C., less than about 20° C., less than about 10° C., less than about 0° C., less than about −10° C., less than about −20° C., less than about −30° C., or any other suitable temperatures.

Optionally, the reaction mixture containing the compound of formula (VII) obtained in step e), before or after conventional work-up, may be carried forward to step f) without isolating the compound.

Step (f) involves converting a compound of formula (VII) in to a compound of formula (VIII). Step (f) may be carried out in the presence of a reagent. Suitable reagent that may be used in step (f) include, but are not limited to: 1,4-diazabicyclo[2.2.2]octane (“DABCO”), 1,8-diazabicyclo[5.4.0]undec-7-ene (“DBU”), triethylamine, tributylamine, N-methylmorpholine, N,N-diisopropylethylamine, N-methylpyrrolidine, pyridine, 4-(N,N-dimethylamino)pyridine, morpholine, imidazole, 2-methylimidazole, 4-methylimidazole, or the like; or any other suitable reagents.

Step (f) may be carried out in a suitable solvent. Suitable solvents that may be used in step f) include, but are not limited to: water; alcohol solvents; ketone solvents; ester solvents; aliphatic or alicyclic hydrocarbon solvents; aromatic hydrocarbon solvents; nitrile solvents; or any mixtures thereof.

Suitable temperatures for the reaction of step (f) may be less than about 100° C., less than about 80° C., less than about 60° C., less than about 40° C., less than about 30° C., less than about 20° C., less than about 10° C., less than about 0° C., or any other suitable temperatures.

Optionally, the reaction mixture containing the compound of formula (VIII) obtained in step f), before or after conventional work-up, may be carried forward to step g) without isolating the compound.

Step (g) involves reacting a compound of formula (VIII) with oxidizing agent to provide a compound of formula (IX).

Step (g) may be carried out in the presence of oxidizing agent. Suitable oxidizing agent that may be used in step g) include, but are not limited to: a complex of pyridine-sulfur trioxide in the presence of diisopropyl ethylamine; a complex of chromium(VI) oxide with pyridine; a complex of chromium trioxide in sulfuric acid; a complex of chromium trioxide with pyridine; or any other suitable oxidizing agents.

Step (g) may be carried out in a suitable solvent. Suitable solvents that may be used in step g) include, but are not limited to: water; halogenated solvents; ether solvents; hydrocarbon solvents; ester solvents; nitrile solvents; aprotic polar solvents, or mixtures thereof.

Suitable temperatures for the reaction of step (g) may be less than about 100° C., less than about 80° C., less than about 60° C., less than about 40° C., less than about 30° C., less than about 20° C., less than about 10° C., less than about 0° C., less than about −10° C., or any other suitable temperatures.

Optionally, the reaction mixture containing the compound of formula (IX) obtained in step g), before or after conventional work-up, may be carried forward to step h) without isolating the compound.

Step (h) involves reacting a compound of formula (IX) with an acid to provide a compound of formula (X).

Step (h) may be carried out in the presence of an acid. Suitable mineral acids that may be used in step h) include, but are not limited to: inorganic acids, such as, for example, hydrochloric acid, sulphuric acid, hydrobromic acid, acetic acid or the like; or any other suitable acids.

Step (h) may be carried out in a suitable solvent. Suitable solvents that may be used in step h) include, but are not limited to: water; nitrile solvents; polar aprotic solvents; ester solvents; ether solvents, or mixtures thereof. Suitable temperatures for the reaction of step (h) may be less than about 100° C., less than about 80° C., less than about 60° C., less than about 40° C., less than about 30° C., less than about 20° C., less than about 10° C., less than about 0° C., or any other suitable temperatures.

Optionally, the reaction mixture containing the compound of formula (X) obtained in step h), before or after conventional work-up, may be carried forward to step i) without isolating the compound.

Step (i) converting a compound of (X) in to lubiprostone of Formula (I).

Step (i) may be carried out in the presence of suitable reducing agents. Suitable reducing agents that may be used in step i) include, but are not limited to: diisobutyl aluminum hydride, Raney nickel, sodium hypophosphate, palladium, or the like; or any other suitable reducing reagents.

Step (i) may be carried out in one or more suitable solvents. Suitable solvents that may be used in step i) include, but are not limited to: ester solvents; ether solvents; aromatic hydrocarbon solvents, or mixtures thereof.

The isolation in step (i) may be effected by methods including removal of solvent, cooling, concentrating the reaction mass, adding an anti-solvent, adding seed crystals, and the like. Suitable temperatures for isolation may be less than about 100° C., or less than about 60° C., or less than about 40° C., or less than about 20° C., or less than about 5° C., or less than about 0° C., or less than about −10° C., or less than about −20° C., or any other suitable temperatures. Suitable times for isolation may be less than about 5 hours, or less than about 3 hours, or less than about 2 hours, or less than about 1 hour, or longer times may be used. The exact temperature and time required for complete isolation may be readily determined by a person skilled in the art and will also depend on parameters, such as, for example, concentration and temperature of the solution or slurry. Stirring or other alternate methods, such as, for example, shaking, agitation, and the like, that mix the contents may also be employed for isolation.

Suitable anti-solvents that may be used include but are not limited to: aliphatic or alicyclic hydrocarbons such as hexanes, n-heptane, n-pentane, cyclohexane, methylcyclohexane, nitromethane or the like; or mixtures thereof.

Suitable techniques that may be used for the removal of solvent include, but are not limited to, rotational distillation using a device, such as, for example, a Büchi Rotavapor, spray drying, agitated thin film drying, freeze drying (lyophilization), and the like, optionally under reduced pressure.

The isolated lubiprostone of formula (I) may be recovered by methods including decantation, centrifugation, gravity filtration, suction filtration, or any other technique for the recovery of solids. The lubiprostone of formula (I) thus isolated may carry some amount of occluded mother liquor and have higher than desired levels of impurities.

The isolated lubiprostone of formula (I) may be further purified by precipitation, slurrying in a suitable solvent, or any other suitable techniques. Precipitation may be achieved by crystallization, such as by cooling a solution, concentrating a solution, or by combining an anti-solvent with a solution of the product, or any other suitable methods. Suitable solvents that may be used for the purification of lubiprostone of formula (I) include, but are not limited to: alcohol solvents, ketone solvents, ester solvents, aromatic hydrocarbons, nitriles or any mixtures thereof, to provide lubiprostone of formula (I) having a purity by HPLC which is essentially pure, substantially pure, or even pure. Suitable anti-solvents include, but are not limited to, aliphatic or alicyclic hydrocarbons such as hexanes, n-heptane, n-pentane, cyclohexane, methylcyclohexane, nitromethane or the like; or any mixtures thereof; or any other suitable anti-solvents.

The recovered solid may be optionally further dried. Drying may be carried out in a tray dryer, vacuum oven, air oven, fluidized bed drier, spin flash dryer, flash dryer, and the like. The drying may be carried out at atmospheric pressure or under a reduced pressure at temperatures of less than about 150° C., or less than about 120° C., or less than about 100° C., or less than about 80° C., or less than about 60° C., or any other suitable temperature as long as the lubiprostone of formula (I) is not degraded in quality. The drying may be carried out for any desired time until the required purity is achieved. For example, it may vary from about 1 to about 10 hours or longer.

The dried product may be optionally milled to get the required particle size. Milling or micronization may be performed before drying, or after the completion of drying of the product. Techniques that may be used for particle size reduction include, without limitation sifting; milling using mills, such as, for example, ball, roller and hammer mills, and jet mills, including, for example, air jet mills; or any other conventional technique.

The desired particle size may also be achieved directly from the reaction mixture by selecting equipment that is able to provide lubiprostone with the desired particle size.

In an aspect, the present application provides a process for the preparation of formula (XII):

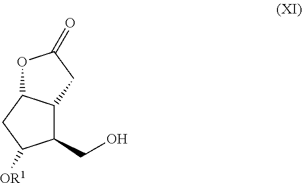

comprising reacting a compound of formula (XI) with a reagent to provide a compound of formula (XII):

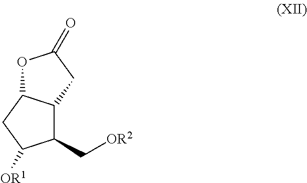

wherein each of R¹ and R² independently is a hydrogen or a protecting group for a hydroxyl group.

Suitable solvents that may be used include, but are not limited to: water; nitrile solvents; polar aprotic solvents; ester solvents; ether solvents; halogenated solvents; aromatic hydrocarbon solvents; aliphatic or acyclic solvents, or mixtures thereof.

The above reaction may be carried out in the presence of a suitable reagent. Suitable reagent that may be used in the in the reaction include, but are not limited to: 2,3-dichloro-5,6-dicyano-1,4-benzoquinone (DDQ), or any other suitable reagent.

### DEFINITIONS

The following definitions are used in connection with the present invention unless the context indicates otherwise.

A “halogenated hydrocarbon solvent” is an organic solvent containing a carbon bound to a halogen. “Halogenated hydrocarbon solvents” include, but are not limited to, dichloromethane, 1,2-dichloroethane, trichloroethylene, perchloroethylene, 1,1,1-trichloroethane, 1,1,2-trichloroethane, chloroform, carbon tetrachloride, or the like.

“Aromatic hydrocarbon solvent” refers to a liquid, unsaturated, cyclic, hydrocarbon containing one or more rings which has at least one 6-carbon ring containing three double bonds. It is capable of dissolving a solute to form a uniformly dispersed solution. Examples of an aromatic hydrocarbon solvent include, but are not limited to, benzene toluene, ethylbenzene, m-xylene, o-xylene, p-xylene, indane, naphthalene, tetralin, trimethylbenzene, chlorobenzene, fluorobenzene, trifluorotoluene, anisole, C₆-C₁₀aromatic hydrocarbons, or mixtures thereof.”

An “ether solvent” is an organic solvent containing an oxygen atom —O— bonded to two other carbon atoms. “Ether solvents” include, but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, C₂₋₆ethers, or the like.

An “ester solvent” is an organic solvent containing a carboxyl group —(C═O)—O— bonded to two other carbon atoms. “Ester solvents” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, C₃₋₆esters, or the like.

A “nitrile solvent” is an organic solvent containing a cyano —(C≡N) bonded to another carbon atom. “Nitrile solvents” include, but are not limited to, acetonitrile, propionitrile, C₂₋₆nitriles, or the like.

An “alcohol solvent” is an organic solvent containing a carbon bound to a hydroxyl group. “Alcohol solvents” include, but are not limited to, methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, C₁₋₆alcohols, or the like.

A “ketone solvent” is an organic solvent containing a carbonyl group —(C═O)— bonded to two other carbon atoms. “Ketone solvents” include, but are not limited to, acetone, ethyl methyl ketone, diethyl ketone, methyl isobutyl ketone, C₃₋₆ketones, and the like.

An “aliphatic or alicyclic hydrocarbon solvent” refers to a liquid, non-aromatic, hydrocarbon, which may be linear, branched, or cyclic. It is capable of dissolving a solute to form a uniformly dispersed solution. Examples of a hydrocarbon solvent include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, C₅-C₈aliphatic hydrocarbons, petroleum ethers, or mixtures thereof.

A “polar aprotic solvent” has a dielectric constant greater than 15 and is at least one selected from the group consisting of amide-based organic solvents, such as N,N-dimethylformamide (DMF), N,N-dimethylacetamide (DMA), N-methylpyrrolidone (NMP), formamide, acetamide, propanamide, hexamethyl phosphoramide (HMPA), and hexamethyl phosphorus triamide (HMPT); nitro-based organic solvents, such as nitromethane, nitroethane, nitropropane, and nitrobenzene; pyridine-based organic solvents, such as pyridine and picoline; sulfone-based solvents, such as dimethylsulfone, diethylsulfone, diisopropylsulfone, 2-methylsulfolane, 3-methylsulfolane, 2,4-dimethylsulfolane, 3,4-dimethy sulfolane, 3-sulfolene, and sulfolane; and sulfoxide-based solvents such as dimethylsulfoxide (DMSO).

“Protecting group for a hydroxyl group” refers to a functional group that is introduced to inactivate the hydroxy group against a specific reaction in order to avoid an undesirable chemical reaction, and as long as it conforms to this purpose, is not limited in particular. For instance, methyl group, methoxy methyl group, ethyl group, 1-ethoxyethyl group, benzyl group, substituted benzyl group, allyl group, tetrahydropyranyl group, t-butyl dimethyl silyl group, triethyl silyl group, triisopropyl silyl group, diphenyl methyl silyl group, formyl group, acetyl group, substituted acetyl group, benzoyl group, substituted benzoyl group, methyloxy carbonyl group, benzyloxy carbonyl group, t-butyloxy carbonyl group, allyloxy carbonyl group, or the like.

All percentages and ratios used herein are by weight of the total composition and all measurements made are at 25° C. and normal pressure unless otherwise designated. All temperatures are in degrees Celsius unless specified otherwise. As used herein, “comprising” means the elements recited, or their equivalent in structure or function, plus any other element or elements that are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. All ranges recited herein include the endpoints, including those that recite a range “between” two values. The terms “about,” “generally,” “substantially,”, and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

The following definitions are used in connection with the present application unless the context indicates otherwise. Celite® is flux-calcined diatomaceous earth. Celite® is a registered trademark of World Minerals Inc. Hyflow is flux-calcined diatomaceous earth treated with sodium carbonate. Hyflo Super Cel® is a registered trademark of the Manville Corp.

Certain specific aspects and embodiments of the present application will be explained in more detail with reference to the following examples, which are provided for purposes of illustration only and should not be construed as limiting the scope of the present application in any manner.

### EXAMPLES

### Example 1

Preparation of (3aR,4R,5R,6aS)-4-((E)-4,4-difluoro-3-oxooct-1-enyl)-2-oxohexahydro-2H-cyclopenta[b]furan-5-yl benzoate. Dess-Martin periodinane (27.63 g) and dichloromethane (120 mL) are charged into a round bottom flask under nitrogen atmosphere and stirred for 5-10 minutes. The reaction mixture is cooled to 0-5° C. A solution of (3aR,4S, 5R,6aS)-4-(hydroxymethyl)-2-oxohexahydro-2H-cyclopenta[b]furan-5-yl benzoate (15 g) in dichloromethane (90 mL) is added to the reaction mixture at 0° C. The reaction mixture is maintained for 5 hours. A solution of sodium thiosulphate pentahydrate (45 g) and sodium bicarbonate (15 g) in water (120 mL) is added to the reaction mixture at 2° C. and maintained for 30 minutes. The reaction mixture is heated to 16° C. and maintained for 30 minutes. Both organic and aqueous layers are separated. The aqueous layer is extracted with dichloromethane (75 mL). The combined organic layer is washed with 10% sodium chloride solution (75 mL). The solvent from the organic layer is evaporated to 10 volumes at 30-35° C. to afford 260 mL of (3aR,4R,5R,6aS)-4-formyl-2-oxohexahydro-2H-cyclopenta[b]furan-5-yl benzoate.

60% Sodium hydride (3.25 g) and tetrahydrofuran (75 mL) are charged in to a round bottom flask under nitrogen atmosphere and stirred for 5-10 minutes. The reaction mixture is cooled to 0-10° C. A solution of dimethyl (3, 3-difluoro-2-oxoheptyl)phosphonate (18.2 g) in tetrahydrofuran (30 mL) is added to the reaction mixture at 0° C. for 30 minutes. The reaction mixture is heated to 26° C. A solution of 0.5 M zinc chloride in THF solution (142 mL) is added to the reaction mixture under nitrogen atmosphere at 26° C. and maintained for 30 minutes. The above obtained solution of (3aR,4R,5R,6aS)-4-formyl-2-oxohexahydro-2H-cyclopenta[b]furan-5-yl benzoate (260 mL) is added to the reaction mixture at 25-28° C. and stirred for 10 minutes. The reaction mixture temperature is raised to 40-45° C. and maintained for 20 hours. The reaction mixture is cooled to 10-15° C. Acetic acid (3.0 mL) is added to the reaction mixture at 10-15° C. and stirred for 10 minutes. The reaction mixture temperature raised to 25-30° C. A solution of ammonium chloride (30 g) in water (150 mL) is added to the reaction mixture at 25° C. for 15 minutes and stirred for 30 minutes. Both organic and aqueous layers are separated. The aqueous layer is extracted with ethyl acetate (75 mL). The combined organic layer is washed with 10% sodium chloride solution (75 mL). The solvent from the organic layer is evaporated completely under reduced pressure at 45° C. The obtained reaction mass is subjected to column chromatography by using ethylacetate/heptane (1:1). The collected fractions are evaporated under reduced pressure at 40-45° C. to afford 14.5 g of the title compound. Purity by HPLC: 93.70%.

### Example 2

Preparation of (3aR,4R,5R,6aS)-4-(4,4-difluoro-3-oxooctyl)-2-oxohexahydro-2H-cyclopenta[b]furan-5-yl benzoate. (3aR,4R,5R,6aS)-4-((E)-4,4-difluoro-3-oxooct-1-enyl)-2-oxohexahydro-2H-cyclopenta[b]furan-5-yl benzoate (81.6 g) and ethyl acetate (520 mL) are charged into a round bottom flask. Activated carbon (40 g) is added to the reaction mixture at 25-30° C. The reaction mixture temperature is raised to 35-40° C. and stirred for 2 hours. The reaction mixture is filtered through hyflow bed and washed with ethyl acetate (300 mL). The resultant filtrate is charged in to a pressure vessel under nitrogen atmosphere. 10% Pd/C (2.44 g) is charged in to the reaction mixture. The reaction mixture is maintained at a pressure of 20-25 psi hydrogen pressure for about 12-16 hours at 20-25° C. The reaction mixture is filtered through Kieselguhr/Celite® bed, washed with ethyl acetate (150 mL) and the solvent from the filtrate is evaporated under reduced pressure at 35-40° C. to afford 80.0 g of the title compound.

### Example 3

Preparation of (3aR,7aR,8aS)-6-(1,1-difluoropentyl)-6-hydroxyoctahydrofuro[3′,2′:3,4]cyclopenta[1,2-b]pyran-2(7aH)-one. Potassium carbonate (1.62 g) and methanol (64 mL) are charged in to a round bottom flask under nitrogen atmosphere at 25° C. and stirred for 10 minutes. A solution of (3aR,4R,5R,6aS)-4-(4,4-difluoro-3-oxooctyl)-2-oxohexahydro-2H-cyclopenta[b]furan-5-yl benzoate (16 g) in methanol (64 mL) is added to the reaction mixture under nitrogen atmosphere at 25° and maintained for 17 hours at 25-35° C. The solvent from the reaction mixture is evaporated under reduced pressure at 38° C. Ethyl acetate (80 mL) is added to the reaction mass and stirred for 15-30 minutes. The solvent from the reaction mixture is evaporated up to 1-2 volumes under reduce pressure at below 40° C. Ethyl acetate (160 mL) is added to the reaction mass and stirred for 20 minutes. The reaction mass is washed with 5% sodium chloride solution (80 mL) and stirred for 10 minutes. The organic layer is separated and the aqueous layer is extracted with ethyl acetate (80 mL). Combined the organic layer and the organic layer is washed with 5% sodium chloride solution (80 mL). The solvent from the organic layer is evaporated completely under reduced pressure at 38° C. The obtained reaction mass is subjected to column chromatography by using 15% acetone/hexane. The collected fractions are evaporated completely under reduced pressure at 45-50° C. The obtained solid is slurred in methyl tertiary butyl ether/heptane (1:3) (80 mL) for 10-15 minutes, filtered, and washed with methyl tertiary butyl ether/heptane (1:3) (32 mL). The solid is dried at 40° C. under vacuum to afford 6.91 g of the title compound. Purity by HPLC: 99.90%.

### Example 4

Preparation of (3aR,4R,5R,6aS)-4-(4,4-difluoro-3-oxooctyl)-5-(tetrahydro-2H-pyran-2-yloxy)hexahydro-2H-cyclopenta[b]furan-2-one. (3aR,7aR,8aS)-6-(1,1-Difluoropentyl)-6-hydroxyoctahydrofuro[3′,2′:3,4]cyclopenta[1,2-b]pyran-2(7aH)-one (39.5 g), dichloromethane (300 mL) and pyridinium p-toluenesulfonate (978 mg) are charged in to a round bottom flask at 25° C. under nitrogen atmosphere and stirred for 10 minutes. 3,4-Dihydro-2H-pyran (27.3 g) is added to the reaction mixture and maintained at 25° C. for 16 hours. Saturated sodium bicarbonate solution (100 mL) is added to the reaction mixture. The organic layer is separated and the aqueous layer is extracted with dichloromethane (50 mL). The organic layer is washed with brine (100 mL), dried over magnesium sulphate (25 g), filtered, and washed with dichloromethane (50 mL). The solvent from the organic layer is evaporated completely under reduced pressure at 35° C. to afford 51.4 g of the title compound.

### Example 5

Preparation of (3aR,4R,5R,6aS)-4-(4,4-difluoro-3-hydroxyoctyl)-5-(tetrahydro-2H-pyran-2-yloxy)hexahydro-2H-cyclopenta[b]furan-2-one. Sodium borohydride (4.99 g) and tetrahydrofuran (180 mL) are charged in to a round bottom flask under nitrogen atmosphere and stirred for 10 minutes. The reaction mixture is cooled to −10° C. and methanol (90 ml) is added to the reaction mixture. The reaction mixture is further cooled to −15° C. and a solution of (3aR,4R,5R,6aS)-4-(4,4-difluoro-3-oxooctyl)-5-(tetrahydro-2H-pyran-2-yloxy)hexahydro-2H-cyclopenta[b]furan-2-one (51.3 g) in tetrahydrofuran (90 ml) is added. The reaction mixture is stirred for 2 hours at −10 to −15° C. The reaction mixture is quenched with saturated ammonium chloride solution (230 ml) and stirred for 10 minutes. Water (65 mL) is added to the reaction mass and stirred for 10 minutes. The reaction mixture is extracted with methyl tertiary butyl ether (2×155 mL). The organic layer is washed with brine (130 mL), dried over magnesium sulphate (25 g), filtered, and washed with methyl tertiary butyl ether (75 mL). The solvent from the organic layer is evaporated completely under reduced pressure at 35° C. to afford 55.6 g of the crude compound. The crude compound is purified by column chromatography using a mixture of dichloromethane and methyl tertiary butyl ether (4:1 ratio) to afford 49.2 g of the title compound.

### Example 6

Preparation of (3aR,4R,5R,6aS)-4-(4,4-difluoro-3-hydroxyoctyl)-5-(tetrahydro-2H-pyran-2-yloxy)hexahydro-2H-cyclopenta[b]furan-2-ol. (3aR,4R,5R,6aS)-4-(4,4-Difluoro-3-hydroxyoctyl)-5-(tetrahydro-2H-pyran-2-yloxy)hexahydro-2H-cyclopenta[b]furan-2-one (44.0 g) and toluene (330 mL) are charged into a round bottom flask under nitrogen atmosphere at 25° C. and stirred for 10 minutes. The reaction mixture is cooled to −68° C. and DIBAL-H (180 mL) is added to the reaction mixture at −65 to −68° C. The reaction mixture is stirred for 2 hours at −65° C. to 70° C. The reaction mixture is quenched with methanol (22 mL). 1M Sulfuric acid (440 mL) is added to the reaction mixture and the mixture was allowed to warm while addition of sulfuric acid to 8° C. The reaction mixture is stirred 20 minutes at 12° C. The organic layer is separated; the aqueous layer is extracted with methyl tertiary butyl ether (2×220 mL). The organic layer is washed with water (220 mL), then with saturated sodium bicarbonate solution (220 mL) and brine (220 mL). The organic layer is dried over magnesium sulphate (30 g), filtered, and washed with methyl tertiary butyl ether (75 mL). The solvent from the organic layer is evaporated completely under reduced pressure at 35-40° C. to afford 47.3 g of the title compound.

### Example 7

Preparation of (Z)-7-((1R,2R,3R,5S)-2-(4,4-difluoro-3-hydroxyoctyl)-5-hydroxy-3-(tetrahydro-2H-pyran-2-yloxy)cyclopentyl)hept-5-enoic acid. (4-Carboxybutyl)triphenylphosphonium bromide (199.8 g) and tetrahydrofuran (265 mL) are charged in to a round bottom flask under nitrogen atmosphere at 25° C. and stirred for 5-10 minutes. The reaction mixture is cooled to 0-5° C. and potassium tert-butoxide (902 mL; 1M in tetrahydrofuran) is added to the reaction mixture. The reaction mixture is stirred for 30 minutes. A solution of (3aR,4R,5R,6aS)-4-(4,4-difluoro-3-hydroxyoctyl)-5-(tetrahydro-2H-pyran-2-yloxy)hexahydro-2H-cyclopenta[b]furan-2-ol (47.3 g) in tetrahydrofuran (175 mL) is added at 0-5° C. and stirred for 2 hours. The reaction mixture is quenched by addition of 1M hydrochloric acid (460 mL) and the organic layer is separated. The solvent from the organic layer is evaporated under reduced pressure at 35-40° C. The aqueous layer is extracted with ethyl acetate (2×350 mL). The above obtained organic layer evaporated reaction mass and ethyl acetate extracts are combined. The lower aqueous phase is separated and the organic phase is washed with water (220 mL) and brine (220 mL). The organic layer is dried over magnesium sulphate (30 g), filtered through silica pad, and washed with ethyl acetate (1760 mL). The solvent from the organic layer is evaporated completely under reduced pressure at 35-40° C. to afford 122.1 g of the title compound.

### Example 8

Preparation of (Z) benzyl 7-((1 R,2R,3R,5S)-2-(4,4-difluoro-3-hydroxyoctyl)-5-hydroxy-3-(tetrahydro-2H-pyran-2-yloxy)cyclopentyl)hept-5-enoate. (Z)-7-((1R,2R,3R,5S)-2-(4,4-Difluoro-3-hydroxyoctyl)-5-hydroxy-3-(tetrahydro-2H-pyran-2-yloxy)cyclopentyl)hept-5-enoic acid (122.1 g) and acetone (440 mL) are charged in to a round bottom flask under nitrogen atmosphere and stirred for 10 minutes. The solvent from the mixture is evaporated at 35-40° C. under reduced pressure. The reaction mass and acetone (400 mL) are charged into a round bottom flask under nitrogen atmosphere and stirred for 10 minutes. 1,8-diazabicyclo[5.4.0] undec-7-ene (DBU; 68.6 g) is added to the reaction mixture at 19-25° C. and stirred for 5-10 minutes. Benzyl bromide (77.1 g) is added to the reaction mixture at 20-25° C. and maintained for 18 hours at 20° C. The solvent from the reaction mixture is evaporated under reduced pressure at 35-40° C. The reaction mass is partitioned between aqueous potassium dihydrogen orthophosphate (61.6 g) in water (790 mL) and ethyl acetate (440 mL). Both layers are separated and the aqueous layer is extracted with ethyl acetate (2×220 mL). Combine the organic layer and washed with water (330 mL) and brine (330 mL). The organic layer is dried over magnesium sulfate (30 g), filtered, and washed with ethyl acetate (100 mL). The solvent from the organic layer is evaporated completely under reduced pressure at 35-40° C. The obtained reaction mass is subjected to column chromatography by using a mixture of ethyl acetate and heptane (2:3). The collected fractions are subjected to evaporation at 35-40° C. to afford 50.9 of the title compound.

### Example 9

Preparation of (Z)-benzyl 7-((1 R,2R,3R)-2-(4,4-difluoro-3-oxooctyl)-5-oxo-3-(tetrahydro-2H-pyran-2-yloxy)cyclopentyl)hept-5-enoate. (Z)-Benzyl 7-((1R,2R,3R,5S)-2-(4,4-difluoro-3-hydroxyoctyl)-5-hydroxy-3-(tetrahydro-2H-pyran-2-yloxy)cyclopentyl)hept-5-enoate (50.8 g) and dichloromethane (380 mL) are charged in to a round bottom flask under nitrogen atmosphere at 28° C. The reaction mixture is cooled to 5° C. and diisopropyl ethyl amine (92.6 g) is added to the reaction mixture at 6-8° C. A solution of sulfur trioxide pyridine complex (57.1 g) in dimethyl sulfoxide (190 mL) is added to the reaction mixture under nitrogen atmosphere at 7-12° C. The reaction mixture is stirred for 2 hours at 12° C. The solvent from the reaction mixture is evaporated under reduced pressure at 35-40° C. up to a level of 80%. The reaction mass is partitioned between methyl tertiary butyl ether (500 mL) and water (250 mL). Both layers are separated and the aqueous layer is extracted with methyl tertiary butyl ether (250 mL). The organic layer is washed with 1M HCl (2×190 mL), water (190 mL), saturated aqueous sodium bicarbonate (190 mL) and brine (190 mL). The organic layer is dried over magnesium sulfate (30 g), filtered, and washed with methyl tertiary butyl ether (100 mL). The solvent from the organic layer is evaporated at 35-40° C. under reduced pressure to afford 49.2 g of the title compound.

### Example 10

Preparation of (Z)-benzyl 7-((2R,4aR,5R,7aR)-2-(1,1-difluoropentyl)-2-hydroxy-6-oxooctahydro-cyclopenta[b]pyran-5-yl)hept-5-enoate. A solution of (Z)-benzyl 7-((1 R,2R,3R)-2-(4,4-difluoro-3-oxooctyl)-5-oxo-3-(tetrahydro-2H-pyran-2-yloxy)cyclopentyl)hept-5-enoate (47.5 g) in acetonitrile (475 mL) is charged in to a round bottom flask at below 20° C. 2M HCl (165 mL) is added to the reaction mixture at 13-16° C. The reaction mixture is stirred at 20° C. for 4 hours. Saturated solution of sodium chloride (330 mL) is added to the reaction mixture. The reaction mixture is extracted with ethyl acetate (2×240 mL). The combined organic layer is washed with saturated aqueous sodium bicarbonate solution (240 mL) and with brine (240 mL). The organic layer is dried over magnesium sulfate (30 g), filtered, and washed with ethyl acetate (100 mL). The solvent from the organic layer is evaporated under reduced pressure at 35-40° C. to afford 46.3 g of the crude compound. The obtained compound is subjected to column chromatography by using 3% methyl tertiary butyl ether in dichloromethane (2.5 L). The collected fractions are evaporated under reduced pressure at 35° C.-40° C. to afford 32.1 g of the title compound.

### Example 11

Preparation of lubiprostone. (Z)-Benzyl 7-((2R,4aR,5R,7aR)-2-(1,1-difluoropentyl)-2-hydroxy-6-oxooctahydro-cyclopenta[b]pyran-5-yl)hept-5-enoate (32.0 g) and ethyl acetate (200 mL) are charged in to a round bottom flask and stirred at 20-30° C. to dissolve (Z)-benzyl 7-((2R,4aR,5R,7aR)-2-(1,1-difluoropentyl)-2-hydroxy-6-oxooctahydro-cyclopenta[b]pyran-5-yl)hept-5-enoate completely. Activated carbon (16.0 g) is added to the above solution at room temperature and stirred for 2 hours. The reaction solution is filtered through Kieselguhr bed and washed with ethyl acetate (150 mL). 10% Pd/C (6.4 g) is charged in to a pressure vessel. The above obtained ethyl acetate solution of (Z)-benzyl 7-((2R,4aR,5R,7aR)-2-(1,1-difluoropentyl)-2-hydroxy-6-oxooctahydro-cyclopenta[b]pyran-5-yl)hept-5-enoate is charged into the pressure vessel at 15-20° C. The reaction mixture is maintained at 17-25° C. and a pressure of 3.0 kg/cm² for about 90 minutes. The reaction mixture is filtered through Kieselguhr pad and washed with ethyl acetate (200 mL). The solvent from the organic layer is evaporated at 35-40° C. under reduced pressure to afford 28.9 g of reaction mass. The reaction mass is dissolved in a mixture of ethyl acetate (10 mL) and pentane (10 mL) and stirred at room temperature. Pentane (200 mL) is slowly added to the mixture with stirring. The reaction mass is stirred for 1 hour at 20-30° C. The obtained solid is collected by filtration, washed with a mixture of ethyl acetate and pentane (1:10, 2×22 mL), and dried under reduced pressure at 35° C. to afford 23.2 g of the title compound.

### Example 12

Purification of lubiprostone. Lubiprostone (21.13 g) and ethyl acetate (31.5 mL) are charged into a reaction vessel and stirred to dissolve lubiprostone completely. Pentane (252 mL) is added slowly to the reaction solution and stirred for 30 minutes. The obtained solid is collected by filtration, washed with a mixture of ethyl acetate and pentane (1:8; 22.5 mL), followed by with pentane (2×20 mL), and dried at 20-25° C. under reduced pressure to afford 19.39 g of the title compound.

### Example 13

Process for the preparation of 4-hydroxy-cyclopent-2-enone. Water (3 Lt) and furfuryl alcohol (150 g) are charged in to the round bottom flask at 27° C. The resultant solution degassed with nitrogen at 27° C. for 60-90 minutes. Potassium dihydrogen phosphate (0.62 g) is charged to the reaction mixture at 27° C. and stirred for 15-20 minutes. 10% phosphoric acid (1.5 mL) is added to the reaction mixture at 27° C. and stirred for 10-20 minutes. The reaction mass is heated to 95-100° C. and maintained for 40 hours. The reaction mixture is cooled to 30° C. Dichloromethane (4×300 mL) is added to the reaction mixture at 30° C. and stirred for 15-30 minutes. The combined organic layer is washed with water (300 mL). Aqueous layer is completely concentrated under high reduced pressure at 60-65° C. Isopropyl alcohol (3×150 mL) is charged in to the reaction mass and evaporated completely at below 65° C. under reduced pressure. The resultant reaction mass is dissolved in dichloromethane (300 mL) and stirred for 10-15 minutes. The obtained solid is separated by filtration and washed with dichloromethane (75 mL). The mother liquor is evaporated to dryness to afford 49.0 g of the title compound. Purity by HPLC: 90.7%

### Example 14

Preparation of cis-cyclopent-4-ene-1,3-diol. 4-Hydroxy-cyclopent-2-enone (100 g), methanol (950 mL) and tetrahydrofuran (950 mL) are charged in to the round bottom flask at 27° C. and stirred for 10 minutes. Cerium (III) chloride heptahydrate (66.78 g), is charged in to the reaction mixture at 27° C. under nitrogen atmosphere and stirred for 30 minutes. The reaction mixture is cooled to −33° C. by using isopropyl alcohol and dry ice. A solution of sodium borohydride (19.26 g) 12.2% of aqueous NaOH solution (20 mL), THF (50 mL) and methanol (50 mL) is slowly added to the reaction mixture at −33° C. and maintained for 1-2 hours. The reaction mixture temperature is raised to the −20° C. Acetic acid (60 mL) is slowly added to the reaction mixture at −19° C. The reaction mixture is heated to 30° C. and maintained for 1-2 hours. The reaction mixture is filtered and washed the wet cake with methanol (100 mL). The resultant filtrate is evaporated at below 55° C. under reduced pressure up to a level of 5-6 volumes. Ethyl acetate (2 Lt) is added to the reaction mass at 27° C. and stirred for 1-2 hours. The reaction mass is filtered through the silica bed (250 g) and washed with ethyl acetate (500 mL). The resultant filtrate is concentrated at below 55° C. under reduced pressure up to 5-6 volumes. The reaction mass is passed through micro filter paper (0.45 micron) two times. The resultant filtrate is concentrated completely at below 55° C. under reduced pressure. Toluene (2×100 mL) is added to the reaction mass and distilled off the solvent completely under reduced pressure at 55° C. to afford 86.0 g of the title compound.

### Example 15

Preparation of (1S,4R)-4-hydroxy-2-cyclopentenyl acetate. Acetone (700 mL), cis-cyclopent-4-ene-1, 3-diol (40 g) and vinyl acetate (68.86 g) are charged in to the round bottom flask at 25° C. under nitrogen atmosphere and stirred for 10 minutes. Lipase PS ‘amano’ IM (1.6 g) is added to the reaction mixture at 25° C. under nitrogen atmosphere and maintained for 48 hours. The reaction mass is filtered through the Celite® bed and wash the bed with acetone (200 mL). The resultant filtrate is concentrated completely at below 55° C. under reduced pressure. The reaction mass is cooled to 25° C. and dissolved in water (180 mL). The aqueous layer is washed with n-heptane (3×200 mL). The aqueous layer charged in to another round bottom flask at 25° C. Sodium chloride (120 g) is added to the reaction mixture. Filtered the undissolved sodium chloride and washed with methyl tertiary butyl ether (MTBE; 240 mL). The aqueous layer is extracted with MTBE (2×200 mL) at 25° C. Combine the organic layer and washed with molecular sieve (40 g). The solvent from the organic layer is evaporated completely under reduced pressure at below 55° C. to afford 31.5 g of the title compound. Purity by HPLC: 86.4%

### Example 16

Preparation of 3,3a,6,6a-tetrahydro-cyclopenta[b]furan-2-one. Triethyl orthoacetate (285.31 g), (1S,4R)-4-hydroxy-2-cyclopentenyl acetate (100 g) and hydroquinone (5 g) are charged in to a round bottom flask at 25° C. and stirred for 15 minutes. The reaction mixture is heated to 112° C. and maintained for 30 minutes. Again the reaction mixture is heated to 144° C. and maintained for 36 hours. The reaction mixture is cooled to 25° C. Water (500 mL) is added to the reaction mixture at 26° C. and stirred for 15 minutes. Ethyl acetate (500 mL) is added to the reaction mixture at 26° C. and stirred for 15 minutes. The solvent from the organic layer is evaporated completely under reduced pressure at below 55° C. The reaction mass is cooled to 25° C. Methanol (500 mL) is added to the reaction mass at 25° C. and stirred for 10 minutes. A solution of potassium hydroxide (98.50 g) in water (500 mL) is added to the reaction mass at 25° C. and stirred for 1-2 hours. The solvent from the reaction mass is evaporated completely under pressure at below 60° C. The reaction mass is washed with ethyl acetate (3×500 mL). The reaction mass pH is adjusted to 1-2 with a solution of concentrated hydrochloric acid. The aqueous layer is extracted with dichloromethane (2×500; 1×300). The solvent from the organic layer is evaporated completely under reduced pressure at below 55° C. to afford 30.7 g of the title compound. Purity by HPLC: 93.87%

### Example 17

Preparation of (3aR,4S,5R,6aS)-5-(acetyloxy)-4-[(acetyloxy)methyl]hexahydro-2H-cyclopenta[b]furan-2-one. Acetic acid (518.43 mL), 3, 3a, 6, 6a-tetrahydro-cyclopenta[b]furan-2-one (125 g), paraformaldehyde (100 g) and acetic anhydride (190.78 mL) are charged in to the round bottom flask at 29° C. and stirred for 10 minutes. Sulphuric acid (21.25 mL) is added slowly to the reaction mixture at 29° C. The reaction mixture is heated to 70° C. and maintained for 35 hours. The reaction mixture is cooled to 50° C. Paraformaldehyde (12.5 g) is added to the reaction mixture at 50° C. The reaction mixture is heated to 79° C. and maintained for 6 hours. The reaction mixture is cooled to 25-30° C. and ethyl acetate (1250 mL) is charged to the reaction mixture. The reaction mixture is cooled to 0-5° C. and a pre cooled solution of potassium hydroxide (496.8 g) in water (2500 mL) is added slowly to the reaction mixture. A solution of sodium bicarbonate (162.5 g) in water (1875 mL) is added slowly to the reaction mass at 10° C. The reaction mixture temperature is raised to 25° C. and stirred for 10 minutes. The reaction mixture is filtered and washed with ethyl acetate (250 mL). Both layers are separated and the organic layer is washed with saturated sodium bicarbonate solution (162.5 g of sodium bicarbonate in 1875 mL of water). The aqueous layer is extracted with ethyl acetate (2×625 mL). The combined organic layer is washed with brine solution. The solvent from the organic layer is evaporated completely under reduced pressure at below to afford 199 g of the title compound.

### Example 18

Preparation of (3aR,4S, 5R,6aS)-hexahydro-5-hydroxy-4-(hydroxymethyl)-2H-cyclopenta[b]furan-2-one. Methanol (500 mL), (3aR,4S, 5R,6aS)-5-(acetyloxy)-4-[(acetyloxy)methyl]hexahydro-2H-cyclopenta[b]furan-2-one (100 g) and Indion 225 H resin (100 g) are charged in to the round bottom flask at 25-35° C. and stirred for 15 minutes. The reaction mixture is heated to 60-65° C. and maintained for 35-40 hours. The reaction mixture is cooled to 25-35° C., filtered through the hyflow bed and washed with methanol (200 mL). The resultant filtrate is concentrated completely under reduced pressure at below 50° C. The obtained reaction mass is subjected to column chromatography by using n-hexane/acetone. The collected fractions are evaporated completely under reduced pressure at 45-50° C. to afford 28.0 g of the title compound. Purity by HPLC: 95.7%

### Example 19

Preparation of (3aR,5R,6aS)-hexahydro-5-hydroxy-4-[(triphenylmethoxy)methyl]-2H-cyclopenta[b]furan-2-one. A solution of (3aR,4S,5R,6aS)-hexahydro-5-hydroxy-4-(hydroxymethyl)-2H-cyclopenta[b]furan-2-one (27 g) solution in tetrahydrofuran (418.5 mL) is charged in to a round bottom flask under nitrogen atmosphere at 27° C. The reaction mixture is heated to 60-65° C. The reaction mixture is cooled to 35-40° C., molecular sieves (13.5 g) is charged to the reaction mixture and stirred for 15-30 minutes. Dimethyl amino pyridine (3.82 g) and triethyl amine (130 mL) are charged to the reaction mixture at 35-40° C. under nitrogen atmosphere. A solution of triphenyl methyl chloride (48 g) in tetrahydrofuran (81 mL) is added to the reaction mixture at 35-40° C. under nitrogen atmosphere. The reaction mixture is heated to 60-65° C. and maintained for 20-24 hours. The reaction mixture is cooled to 25-35° C., filtered through the hyflow bed and washed with ethyl acetate (81 mL). The resultant filtrate is washed with water (270 mL). The aqueous layer is extracted with ethyl acetate (2×135 mL). The combined organic layer is washed with water (135 mL). The solvent from the organic layer is evaporated completely under reduced pressure at below 55° C. to afford 61.5 g of the title compound. Purity by HPLC: 68%.

### Example 20

Preparation of (3aR,4S,5R,6aS)-5-(benzoyloxy)hexahydro-4-[(triphenylmethoxy)methyl]-2H-cyclopenta[b]furan-2-one. (3aR,5R,6aS)-Hexahydro-5-hydroxy-4-[(triphenylmethoxy)methyl]-2H-cyclopenta[b]furan-2-one (28 g), dichloromethane (280 mL), triethylamine (10.26 g) and dimethyl aminopyridine (1.65 g) are charged in to the round bottom flask at 27° C. under nitrogen pressure and stirred for 10 minutes. A solution of benzoyl chloride (11.40 g) in dichloromethane (56 mL) is added drop wise to the reaction mixture at below 40° C. for 15-30 minutes under nitrogen atmosphere. The reaction mixture is heated to 35-40° C. and maintained for 2-4 hours. The reaction mixture is cooled to 25-35° C. 5% sodium bicarbonate solution (280 mL) is charged to the reaction mixture at 25-35° C. and stirred for 15-30 minutes. Both layers are separated, the organic layer is washed with 5% sodium bicarbonate solution (280 mL) and water (280 mL). The solvent from the organic layer is evaporated under reduced pressure at below 45° C. Ethyl acetate (100.8 mL) is added to the reaction mass at below 45° C. and stirred for 5-10 minutes. The reaction mixture is heated to 50-60° C. and stirred for 15-30 minutes. The reaction mixture is cooled to 35-45° C., n-hexane (336 mL) is slowly added to the reaction mass and stirred for 15-30 minutes. The reaction mixture is cooled to 25-35° C. and stirred for 3-5 hours. The obtained solid is collected by filtration, washed with a mixture of ethyl acetate and hexane (56 mL; 1:9), and dried under reduced pressure at 55° C. to afford 18.0 g of the title compound. Purity by HPLC: 97.9%

### Example 21

Preparation of (3aR,4S,5R,6aS)-4-(hydroxymethyl)-2-oxohexahydro-2H-cyclopenta[b]furan-5-yl benzoate. (3aR,4S,5R,6aS)-5-(Benzoyloxy)hexahydro-4-[(triphenylmethoxy)methyl]-2H-cyclopenta[b]furan-2-one (20 g) and dichloromethane (300 mL) are charged in to the round bottom flask at 29° C. and stirred for 10 minutes. Charcoal (5 g) is charged to the reaction mixture at 29° C. The reaction mixture is heated to 35-40° C. and maintained for 1 hour. The reaction mixture is cooled to 25-30° C., silica (40 g) is charged to the reaction mixture, and stirred for 25 minutes. The reaction mixture is filtered through the hyflow bed and washed with dichloromethane (3×100 mL). The silica gel slurry is stirred in dichloromethane (2×100 mL) and filtered. The combined dichloromethane filtrates are concentrated under reduced pressure at below 40° C. Acetonitrile (240 mL) and water (40 mL) are charged in to the reaction mass at 30° C. A solution of 2,3-dichloro,5,6-dicyano-1,4-benzoquinone (DDQ; 0.875 g) in acetonitrile (60 mL) is added to the reaction mixture at 30° C. and stirred for 10 minutes. The reaction mixture is heated to 60° C. and maintained for 2 hours. The reaction mixture is cooled to 25-30° C. Water (450 mL) is added to the reaction mass at 30° C. and stirred for 1 hour. The reaction mixture is cooled to 15-20° C. and stirred for 1 hour. The reaction mixture is filtered and washed with water (40 mL). The resultant filtrate is concentrated at below 50° C. under reduced pressure up to reaction mixture volume reaches to 500 mL The reaction mass is cooled to 25-30° C. Sodium chloride (10 g) is added to the reaction mass at 30° C. and stirred for 10 minutes. Ethyl acetate (1×200 mL; 2×100 mL) is added to the reaction mass at 30° C. and stirred for 10 minutes. The combined organic layer is washed with 5% sodium bicarbonate solution (200 mL) and 1% brine solution (200 mL). The aqueous layer is extracted with ethyl acetate (100 mL). The organic layer is washed with 1% brine solution (100 mL). The combined organic layer is charged in to round bottom flask at 30° C. Norit supra charcoal (5 g) is charged to the reaction mixture at 30° C. The reaction mixture is heated to 50-60° C. and maintained for 1 hour. The reaction mixture is cooled to 25-35° C. and filtered through the hyflow bed and washed with ethyl acetate (60 mL). The resultant filtrate is concentrated at below 50° C. under reduced pressure. Ethyl acetate (60 mL) is charged to the reaction mass and heated to 50° C. Hexane (240 mL) is added slowly to the reaction solution at 50° C. The reaction mass is cooled to 25-30° C. and maintained for 1 hour. The obtained solid is collected by filtration, washed with a mixture of ethyl acetate and hexane (60 mL; 1:4), and dried under reduced pressure at 55° C. for 6 hours to afford 7.4 of the title compound. Purity by HPLC: 99.9%.

1. A process for the preparation of lubiprostone of formula (I): 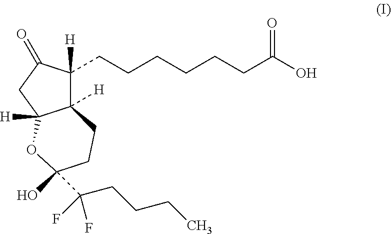
which comprises:
(a) reacting a compound of formula (II) with a reagent to provide a compound of formula (III); 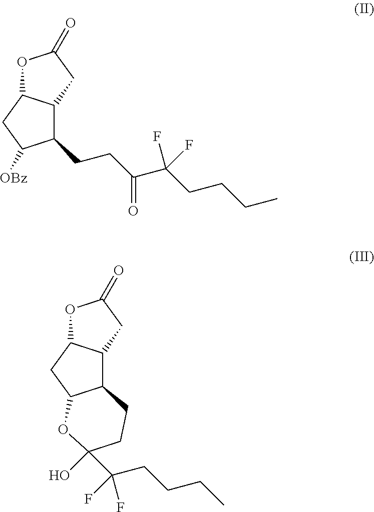
(b) reacting the compound of formula (III) with dihydropyran to provide a compound of formula (IV); 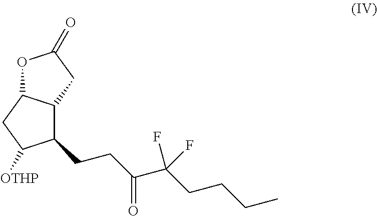
(c) converting a compound of formula (IV) in to a compound of formula (V); 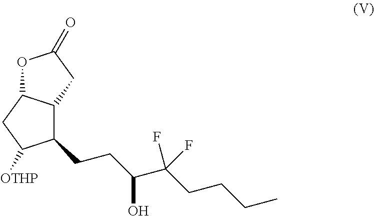
(d) converting a compound of formula (V) in to a compound of formula (VI); 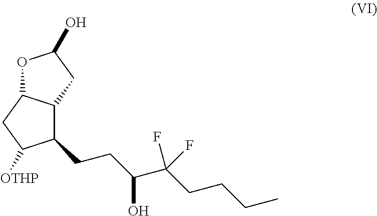
(e) reacting a compound of formula (VI) with (4-carboxybutyl) triphenylphosphonium bromide to provide a compound of formula (VII); 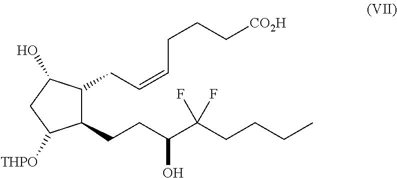
(f) converting a compound of formula (VII) in to a compound of formula (VIII); 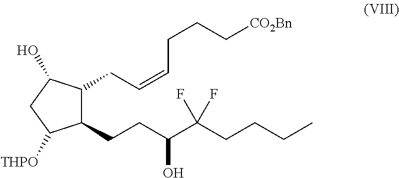
(g) reacting a compound of formula (VIII) with oxidizing agent to provide a compound of formula (IX); 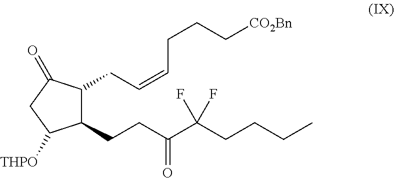
(h) reacting a compound of formula (IX) with an acid to provide a compound of formula (X); and 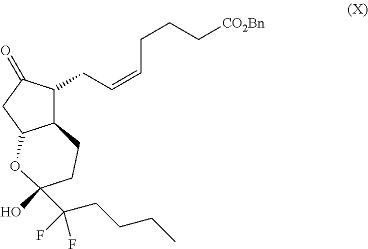
(i) converting a compound of formula (X) in to lubiprostone of formula (I).

2. The process of claim 1, wherein the reagent in step (a) comprises one or more of sodium carbonate, potassium carbonate, lithium carbonate and cesium carbonate.

3. The process of claim 1, wherein the solvent comprises any one or more of methanol, ethanol, 1-propanol and 2-propanol.

4. The process of claim 1, wherein the reagent in step (b) comprises one or more of pyridinium-p-toluenesulfonate, pyridinium dichromate, carbonyldiimidazole and dicyclohexylcarbodiimide.

5. The process of claim 1, wherein the oxidizing agent in step (g) comprises pyridine-sulfur trioxide.

6. The process of claim 1, wherein the acid in step (h) comprises one or more of hydrochloric acid, hydrobromic acid, and sulfuric acid.
